(19) United States
(12) Patent Application Publication
(10) Pub. No.: US 2024/0197659 A1
(43) Pub. Date: Jun. 20, 2024

(54) METHODS FOR TREATING SYMPTOMS OF KABUKI SYNDROME

(71) Applicant: UNIVERSITY OF SOUTH FLORIDA, Tampa, FL (US)

(72) Inventors: Dominic D'AGOSTINO, Plant City, FL (US); Sara E. MOSS, Brooksville, FL (US); Angela Marie POFF, Tampa, FL (US)

(21) Appl. No.: 18/556,969

(22) PCT Filed: Apr. 26, 2022
(86) PCT No.: PCT/US2022/026294

§ 371 Date: Oct. 24, 2023

Related U.S. Application Data
(60) Provisional application No. 63/179,777, filed on Apr. 26, 2021.

(51) Int. Cl. A61K 31/19; A61K 31/20; A61P 3/00

(52) U.S. Cl. CPC A61K 31/19; A61K 31/20; A61P 3/00

(57) ABSTRACT

This disclosure provides methods of treating medical disorders, and more particularly methods for treating one or more symptoms of Kabuki Syndrome.

### CROSS-REFERENCE TO RELATED APPLICATIONS

This application claims the benefit of priority to United States Provisional Application No. 63/179,777, filed Apr. 26, 2021, the disclosure of which is incorporated herein by reference in its entirety.

### TECHNICAL FIELD

This disclosure relates to methods of treating medical disorders, and more particularly to methods for treating one or more symptoms of Kabuki Syndrome.

### BACKGROUND

Kabuki Syndrome is a rare genetic disorder that leads to many developmental abnormalities. Kabuki Syndrome is caused by a heterozygous mutation in either KMT2D (Type 1) or KDM6A (Type 2) that leads to a loss of function. Both genes play a role in gene regulation via histone modification. KMT2D is a gene that codes for the protein kmt2d, a lysine methyltransferase responsible for methylating H3K4. The KDM6A gene encodes the protein kdm6a, a demethylase responsible for demethylating H3K27. Both gene functions contribute to the opening of the chromatin. When either of these two proteins are deficient, craniofacial, skeletal, mental, and dermatological development are severely impacted. Other developmental processes are impacted, but to varying degrees across patients.

There is a clear need for potential therapeutics which may prove useful in the treatment of symptoms associated with Kabuki Syndrome.

### SUMMARY

The present disclosure provides methods for treating, such as mitigating, attenuating, or ameliorating, one or more symptoms of Kabuki Syndrome in a subject in need thereof.

In one aspect, a method is provided of treating one or more symptoms of Kabuki Syndrome in a subject in need thereof, the method comprising administering a therapeutically effective amount of a ketogenic composition comprising a ketogenic compound.

In another aspect, a method is provided to attenuate, mitigate, or ameliorate one or more symptoms of Kabuki Syndrome in a subject in need thereof, the method comprising administering a therapeutically effective amount of a ketogenic composition comprising a ketogenic compound.

A method is also provided of treating one or more symptoms of Kabuki Syndrome in a subject in need thereof comprising administering a therapeutically effective amount of a ketogenic composition to the subject, wherein the ketogenic composition comprises a beta-hydroxybutyrate (BHB) and a medium chain triglyceride (MCT).

A ketogenic composition comprising a ketogenic compound and a medium chain triglyceride (MCT) is also provided for use in treating one or more symptoms of Kabuki Syndrome in a subject in need thereof.

Use of a ketogenic compound and a medium chain triglyceride (MCT) in the manufacture of a ketogenic composition for treating one or more symptoms of Kabuki Syndrome in a subject in need thereof is also provided.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1F provide data comparing Kabuki mice to Wild-Type mice. Groups: Kabuki Female Standard Diet (MutFemaleSD); Wild Type Female Standard Diet (WTFemaleSD); (FIG. 1A) mutated mice showed lower glucose levels than wild type mice. (FIG. 1B) Mutated mice have similar blood ketone levels compared to wild type. (FIG. 1C) Mutated mice have a lower EOL body weight than wild type. (FIGS. 1D and 1E) Mutated mice have similar grip strengths compared to wild type. (FIG. 1F) EOL heart weight is less in the mutated mice compared to wild type.

FIGS. 2A-2I show data comparing Kabuki mice to Wild-Type Mice. Groups: Kabuki Female Standard Diet (MutFemaleSD): Wild Type Female Standard Diet (WTFemaleSD); (FIG. 2A) Mutated mice showed slightly lower lung weights than wild type mice. (FIG. 2B) Mutated mice have similar brain weight compared to wild type. (FIGS. 2C and 2D) Mutated mice have a lower EOL femur length and a larger tibia length than Wild type. (FIGS. 2E and 2F) Mutated mie spent similar amounts of time in the open and closed arms of the elevated plus maze compared to wild type. (FIGS. 2G, 2H, and 2I) Mutated mice spent more time in the center and less time along the perimeter of the open field compared to wild type.

FIGS. 3A-3F provide data comparing BHB treated mice to untreated mice. Groups: Wild Type Female BHB treated (WTFemaleTX); Wild Type Female Standard Diet (WTFemaleSD); (FIG. 3A) Treated mice showed similar blood glucose levels compared to untreated mice. (FIG. 3B) Treated mice have higher blood ketone levels compared to non-treated mice. (FIG. 3C) Treated mice have a similar EOL body weight compared to untreated. (FIGS. 3D and 3E) Treated mice have slightly higher grip strengths compared to untreated. (FIG. 3F) EOL heart weight is less in the treated mice compared to untreated.

FIGS. 4A-4I provide data compared BHB treated mice to untreated mice. Groups: Wild Type Female BHB Treated (WTFemaleTX); Wild Type Female Standard Diet (WTFemaleSD); (FIG. 4A) Treated mice showed similar lung weight compared to untreated mice. (FIG. 4B) Treated mice have higher brain weight compared to untreated. (FIGS. 4C and 4D) Treated mice have a similar EOL femur length and tibia length compared to untreated. (FIGS. 4E and 4F) Treated mice spent less time in the closed arms and more time in the open arms of the elevated plus maze compared to untreated. (FIGS. 4G, 4H, and 4I) Treated mice spent similar amounts of time in the center and along the perimeter of the open field compared to untreated.

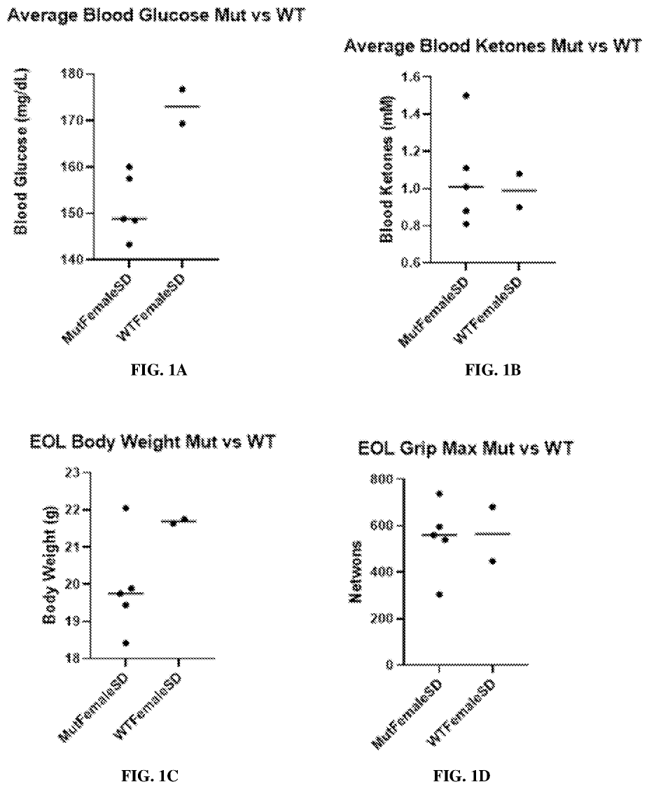

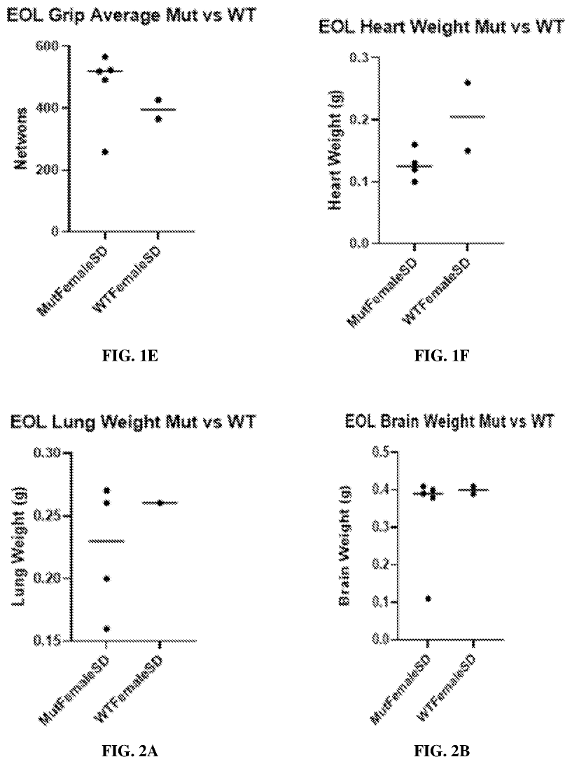

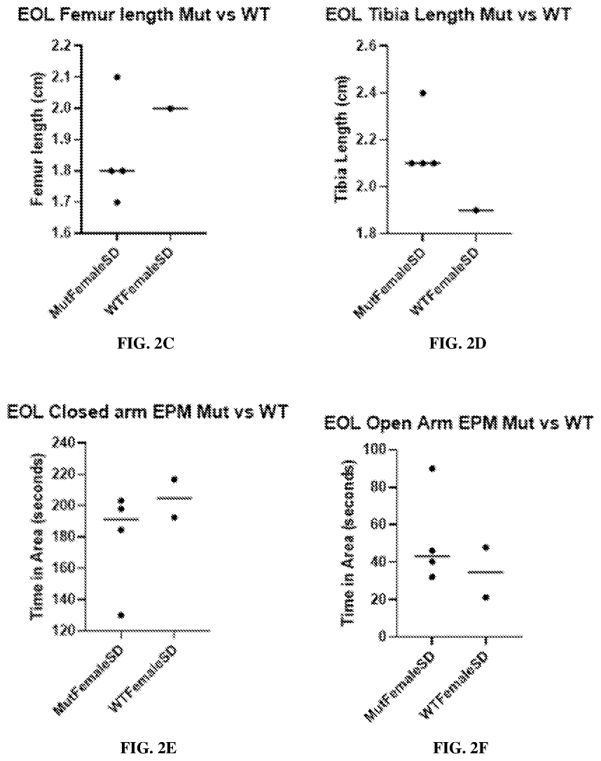

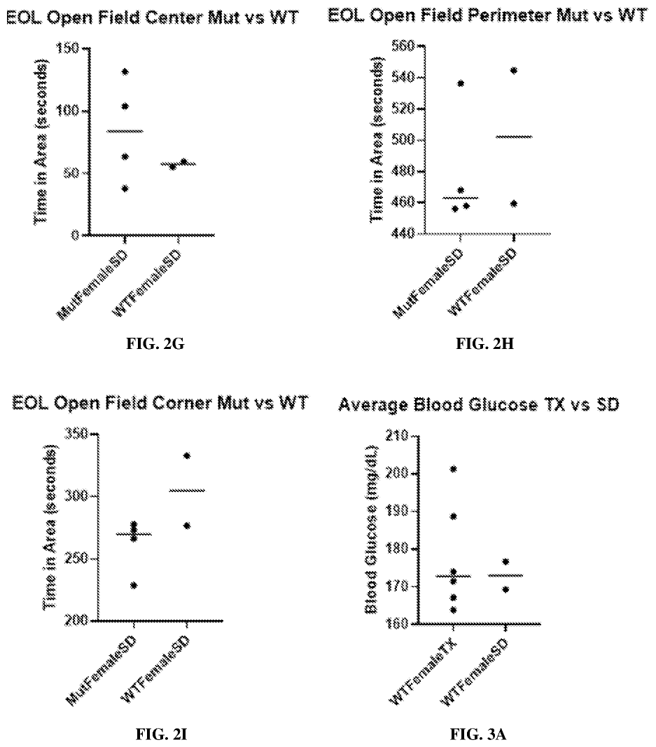

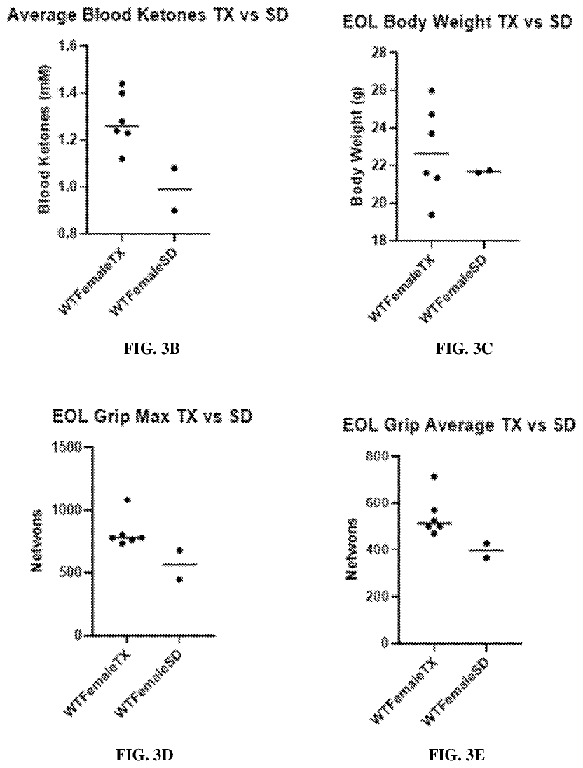

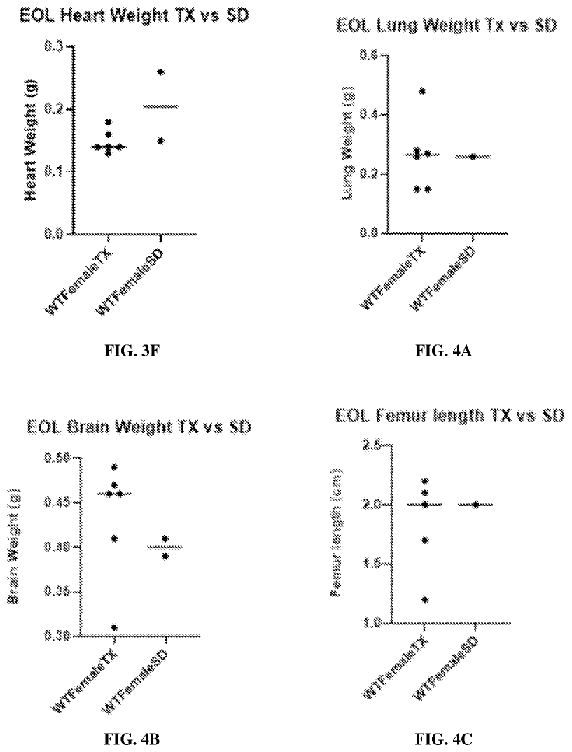

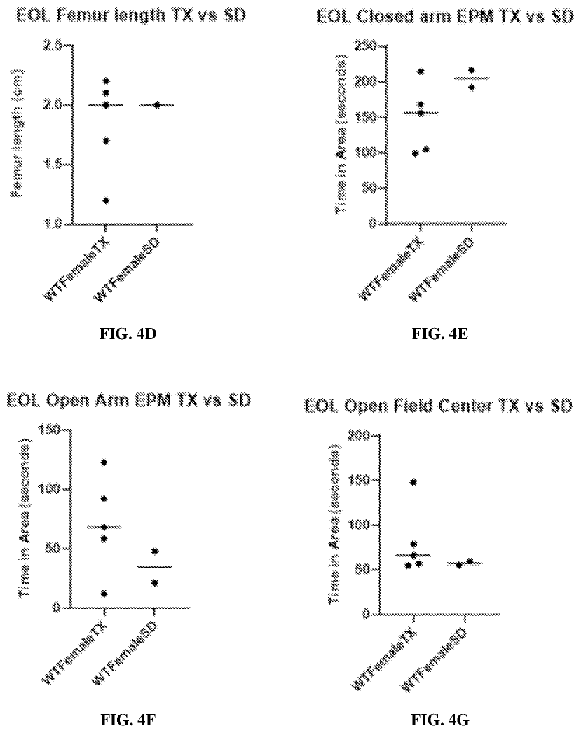

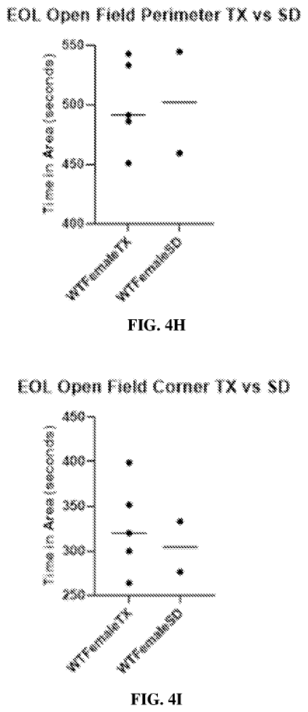

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a symptom”, includes, but is not limited to, two or more such compounds, compositions, or symptoms, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as Kabuki syndrome. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

The term “ketogenic composition” as used herein refers to a composition comprising one or more ketogenic compounds.

“Ketogenic compound” refers to a compound that is capable of elevating ketone body concentrations in a subject. The ketogenic compound can be derived from, for example, a ketone body precursor, a ketone ester, a ketone salt, or combinations thereof.

“Ketone” or “ketone body”, as used interchangeably herein, refers to a compound or species which is selected from O-hydroxybutyrate (OHB), acetoacetate, acetone, or a combination thereof. A ketone body may be derived from a “ketone body precursor”, that is, a compound or species which is a precursor to a ketone body and which may be converted or metabolized to a ketone body in a subject.

“Ketone body ester” or “ketone ester” as used herein, refer to an ester of a ketone body, ketone body precursor, or derivative thereof. Any suitable ketone ester known in the art may be used.

“Ketone body salt” or “ketone salt” is a salt of a ketone body, ketone body precursor, or derivative thereof. The ketone salt may be combined with one or more monovalent cations, divalent cations, or alkaline amino acids. Any suitable ketone salt known in the art may be used.

The terms “beta-hydroxybutyrate”, “βHB”, or “BHB” as used interchangeably herein refer to a carboxylic acid having the general formula CH₃—CHOH—CH₂—C(═O)OH. βHB is a ketone body which may be utilized by the body as a fuel source during instances of low glucose levels.

The terms “ketogenic state” or “ketosis” as used interchangeable herein, refer to a subject having blood ketone body levels within the range of about 0.5 mmol/L to about 10 mmol/L. Levels above 10 mmol/L are associated with ketoacidosis. Ketosis may be achieved in a subject by administering a ketogenic diet or a composition including a ketogenic compound.

“Ketogenic diet” as used herein refers to a diet that causes a metabolic switch from burning glucose for energy to burning fats for energy. Nutritional ketosis or a ketogenic state may be achieved through calorie restriction, fasting, prolonged exercise, and/or a ketogenic diet that is high in fat and restricted in carbohydrates (e.g., sugars).

“Non-ketogenic diet” as used herein refers to a diet that is not capable of achieving ketosis or a ketogenic state in a subject.

The term “medium chain triglycerides” (MCT) as used herein refers to molecules having a glycerol backbone attached to three medium chain fatty acids. Medium chain fatty acids range from 6 to 12 carbon atoms in length. Exemplary medium chain fatty acids include, but are not limited to, caprylic acid, also known as octanoic acid, comprising 8 carbon atoms, and capric acid, also known as decanoic acid, comprising 10 carbon atoms.

The present disclosure provides methods to treat, such as attenuate, mitigate, or ameliorate, one or more symptoms of Kabuki syndrome. Kabuki syndrome (also known as Kabuki-makeup syndrome (KMS) or Niikawa-Kuroki syndrome) is a congenital disorder of genetic origin which affects multiple parts of the body with varying symptoms and severity. Kabuki syndrome is quite rare, affecting roughly one in 32,000 births. The most common feature is a characteristic facial appearance, and it was named Kabuki syndrome because of the facial resemblance of the affected individuals to stage makeup used in kabuki, a Japanese traditional theatrical form.

Type 1 Kabuki syndrome is caused by germline heterozygous loss of function variants in KMT2D (also known as MLL2), located on human chromosome 12. It is estimated that between 55 and 80% of cases of Kabuki syndrome are of Type 1. Type 1 Kabuki syndrome demonstrates an autosomal dominant pattern of inheritance. Type 2 Kabuki syndrome is caused by germline hemizygous (in males) or heterozygous (in females) chromosome deletions or loss of function point variants involving KDM6A, located on the X chromosome. Approximately 5% of cases of Kabuki syndrome are Type 2. Type 2 Kabuki syndrome demonstrates an X-linked dominant pattern of inheritance. Most cases of Kabuki syndrome occur de novo, that is, the parents are unaffected, and the gene was mutated early in embryological development. However, several cases of inherited mutations causing Type 1 or Type 2 Kabuki syndrome are now known. About 30% of cases have no identifiable causative mutation. These may represent individuals with types of mutations that are challenging to detect via current routine investigations or patients who have disorders that share some features with Kabuki syndrome.

The KMT2D and KDM6A genes belong to a family of genes called chromatin-modifying enzymes. Specifically, these genes code for a histone methyltransferase (KMT2D) and a histone demethylase (KMD6A) and play a part in the regulation of gene expression. Under normal circumstances, these enzymes transfer methyl groups on and off histones to regulate genes via epigenetic pathways. When the genes that encode these enzymes are mutated, epigenetic activation of certain developmental genes is impaired and developmental abnormalities occur, leading to the characteristics of Kabuki syndrome patients. The specific developmental genes that are affected by the impaired epigenetic mechanisms in Kabuki syndrome are not yet fully known. There are hundreds of different mutations that have been identified in Kabuki syndrome patients. Most of these mutations are in the KMT2D gene and involve a change in amino acid sequence that creates a shortened and nonfunctional chromatin-modifying enzyme.

Specific symptoms for Kabuki syndrome vary, with large differences between affected individuals. Most people with Kabuki syndrome have distinctive facial features that include arched eyebrows, long eyelashes, elongated eyelids with lower lids that turn out, prominent ears, a flat tip of the nose, and a downward slant to the mouth. Overlapping phenotypic features for patients between KDM6A and KDM6B variations include prominent ears, abnormal dentition, congenital heart disease, feeding difficulties, cryptorchidism, joint hypermobility, developmental delay, hypotonia, and behavioral difficulties. Other common symptoms include skeletal abnormalities, short stature, heart defects, feeding difficulties and a failure to thrive, vision and hearing difficulties, weak muscle tone (hypotonia), small head size (microcephaly), and frequent infections. Mild to moderate intellectual disability and mild to severe developmental delay are often associated with Kabuki syndrome. Infants and young children often experience difficulties relating to hypotonia, feeding issues/failure to thrive, infections, surgical repair of heart and palate defects, and developmental delays. Young children with Kabuki syndrome benefit from early intervention services. School age children tend to have fewer medical issues requiring hospitalization, though frequent infections, hearing loss, and feeding issues occur. In addition, intellectual impairment, difficulty with visuospatial tasks and maintaining attention usually require an individualized education plan if the child attends public school. Older children and adults report difficulties with anxiety. Endocrine abnormalities and immune system abnormalities such as idiopathic thrombocytopenia (ITP) and common variable immune deficiency (CVID) are medical issues that tend to present in older children, adolescents and adults.

In one aspect, a method is provided for treating one or more symptoms of Kabuki syndrome in a subject in need thereof, the method comprising administering to the subject a therapeutically effective amount of a ketogenic composition comprising a ketogenic compound as described herein. In another aspect, methods are provided to mitigate, attenuate, or ameliorate one or more symptoms of Kabuki syndrome in a subject in need thereof, the method comprising administering a therapeutically effective amount of a ketogenic composition comprising a ketogenic compound.

Representative symptoms of Kabuki syndrome which may be mitigated, attenuated, or ameliorated by the methods described herein include, but are not limited to: long eyelashes, depressed nasal tip, atypical fingerprints, ear deformity (such as macrotia or microtia), hypotonia, joint hyper-flexibility, ptosis, blue sclera, café au lait spot, genitourinary abnormalities (such as hypospadias or horseshoe kidney), gastrointestinal abnormalities (such as anal atresia or intestinal malformation), hearing loss, immune deficiencies (such as hypogammaglobinemia), feeding difficulties (in infants), obesity (in adult), short stature, poor sleep, hyperinsulinemia, hypoglycemia, epilepsy, cardiac defects (such as coarctation of the aorta), vertebral anomalies (such as butterfly vertebrae), sparse lateral eyelash, finger anomalies (such as short fifth finger), cleft palate, dental issues, precocious puberty, scoliosis, and hip dysplasia.

In the methods described herein, a ketogenic composition comprising one or more ketogenic compounds can be administered. In some embodiments, the ketogenic compound can be any compound capable of elevating ketone body concentrations in a subject. For example, the ketogenic compound can elevate expression of βHB following administration to the subject. The ketone compound can be a ketone body precursor, a ketone ester, a ketone salt, or a combination thereof. For example, the ketogenic compound can be a ketone body precursor or derivative thereof. Any suitable ketone body precursor which can be metabolized into a ketone body upon administration to the subject can be used. For example, the ketogenic compound can be 1,3-butanediol, acetoacetate, or βHB moieties or derivatives thereof, including esters and salts thereof. For example, the ketogenic compound can be 1,3-butanediol-acetoacetate diester. In some embodiments, the ketogenic compound can be sodium-3-hydroxybutyrate. In some embodiments, the ketogenic compound can be R,S-sodium-3-hydroxybutyrate.

In some embodiments, the ketogenic compound can include a ketone ester. Suitable ketone esters can be prepared using any suitable physiologically compatible alcohol.

Examples of polyhydric alcohols suitable for preparing such esters include carbohydrates and carbohydrate derivatives, such as carbohydrate alcohols. Examples of suitable carbohydrates include, but are not limited to, altrose, arabinose, dextrose, erythrose, fructose, galactose, glucose, gulose, idose, lactose, lyxose, mannose, ribose, sucrose, talose, threose, xylose, and the like. In some embodiments, the ketone ester can be a monoester, a diester, a polyester, or any combination thereof. In some embodiments, the ketone ester can be a diester. In some embodiments, the ketone ester can be a polyester. In some embodiments, the ketone ester can be a glycerol monoester or diester. In some embodiments, the monoester may be esterified at the 1 position. In some embodiments, the diester may be esterified at the 1 and 3 positions. In some embodiments, the ketone ester may comprise a monoester of butane-1,3-diol with D-3-hydroxybutyrate or L-3-hydroxybutyrate, for example 3-hydroxybutyl-L,D-P-hydroxybutyrate, or a diester of glycerol with D-3-hydroxybutyrate or L-3-hydroxybutyrate. The ester may be in an enantiomerically enriched form. In some embodiments, the ketone ester can be a monoester. In some embodiments, for example, the ketone ester can be 1,3-butanediol-acetoacetate monoester, 1,3-butanediol-acetoacetate diester, or combinations thereof.

In some embodiments, the ketogenic compound can be a ketone salt. The ketone salt can be combined with a monovalent cation, divalent cation, or alkaline amino acid. Any suitable ketone salt can be used. For example, the ketone salt can be a βHB salt. In some embodiments, the ketone salt can be a βHB mineral salt. For example, the βHB mineral salt can be potassium βHB, sodium βHB, calcium βHB, magnesium βHB, lithium βHB, or any other feasible non-toxic mineral salts of βHB. In some embodiments, the ketone salt can be a βHB organic salt. Organic salts of βHB include salts of organic bases such as arginine βHB, lysine βHB, histidine βHB, ornithine βHB, creatine βHB, agmatine βHB, or citrulline βHB. The ketone salt can be a combination of βHB. For example, the ketone salt can be a sodium/potassium βHB mineral salt. (Na⁺K⁺βHB). In some embodiments, the ketone salt can be a sodium/calcium βHB mineral salt (Na⁺Ca²⁺βHB). The ketone salt can be mixed into a solution. For example, a βHB mineral salt can be mixed into a solution. The βHB mineral salt can be from 1 to 99% by weight of a solution. For example, the βHB mineral salt can be about 5-95%, about 10-90%, about 20-80%, about 30-70%, about 40-60%, or about 50% by weight of a solution.

In some embodiments, the ketogenic composition can further include a medium chain fatty acid or ester thereof. In some embodiments, the composition can include MCT oil. Sources of the medium chain fatty acid or an ester thereof include coconut oil, coconut milk powder, fractionated coconut oil, palm oil, palm kernel oil, caprylic acid, isolated medium chain fatty acids such as isolated hexanoic acid, isolated octanoic acid, isolated decanoic acid, medium chain triglycerides either purified or in a natural form such as coconut oil and ester derivatives of the medium chain fatty acids such as ethoxylated triglyceride, enone triglyceride derivatives, aldehyde triglyceride derivatives, monoglyceride derivatives, diglyceride derivatives, and triglyceride derivatives, or salts of the medium chain triglycerides. Ester derivatives can optionally include alkyl ester derivatives, such as methyl, ethyl, propyl, butyl, and hexyl. Derivatives can be prepared by any process known in the art, such as direct esterification, rearrangement, fractionation, transesterification, or the like.

In some embodiments, the ketogenic composition can include a ketone ester, a ketone salt, a ketone body precursor, and a medium chain fatty acid. In some embodiments, the ketogenic composition can include a ketone ester, a ketone salt, and a ketone body precursor. In some embodiments, the ketogenic composition can include a ketone ester and a ketone salt. In some embodiments, the ketogenic composition can include a ketone salt, a ketone ester, and medium chain fatty acid. In some embodiments, the ketogenic composition can include a ketone salt and a medium chain fatty acid. In some embodiments, the ketogenic composition can include a ketone ester and a medium chain fatty acid.

In some embodiments, the ketogenic composition can include a ketone salt, a ketone body precursor, and a medium chain fatty acid. In some embodiments, the ketogenic composition can include a ketone ester, a ketone body precursor, and a medium chain fatty acid. In some embodiments, the ketogenic composition can include a ketone body precursor and a medium chain fatty acid.

In some embodiments, the ketogenic composition can include a ketone salt, a ketone body precursor, and a medium chain fatty acid. In some embodiments, the ketogenic composition can include a ketone ester, a ketone body precursor, and a medium chain fatty acid. In some embodiments, the ketogenic composition can include a ketone body precursor and a medium chain fatty acid.

For example, the ketogenic composition can include a sodium/calcium or sodium/potassium βHB mineral salt and a 1,3-butanediol acetoacetate diester. The ketogenic composition can include a sodium/calcium or sodium/potassium βHB mineral salt and a MCT. The composition can include a ketone ester and a medium chain fatty acid. For example, the composition can include 1,3-butanediol acetoacetate diester and a MCT. The composition can include a ketone salt and a MCT mixed at about a 1:1 ratio. The composition can include a ketone ester and a MCT mixed at about a 1:1 ratio. The composition can include a ketone body precursor and a MCT mixed at about a 1:1 ratio. In some embodiments, the MCT can include 65% caprylic triglyceride. In some embodiments, the MCT can include 60% caprylic triglyceride and 40% capric triglyceride.

In some embodiments, the ketogenic composition can comprise a beta-hydroxybutyrate and an MCT. In some embodiments, the ketogenic composition can include a beta-hydroxybutyrate and an MCT mixed at about a 1:1 ratio. In some embodiments, the MCT can include 65% caprylic triglyceride. In some embodiments, the MCT can include 60% caprylic triglyceride and 40% capric triglyceride. In some embodiments, the beta-hydroxybutyrate and the MCT are present in the ketogenic composition in an amount from about 5% to about 50% by weight of the ketogenic composition, for example in about 5%, about 10%, about 15%, about 20%, about 25%, about 30%, about 35%, about 40%, about 45%, or about 50% by weight of the composition.

In some embodiments, the beta-hydroxybutyrate and the MCT are present in an amount from about 5% to about 25% by weight of the composition. In particular embodiments, the beta-hydroxybutyrate and the MCT are present in the ketogenic composition in an amount of about 15% by weight of the composition.

The ketogenic composition can further include other nutritional substrates. For example, the ketogenic composition can further include free amino acid, amino acid metabolites, vitamins, minerals, electrolytes and metabolic optimizers such as NADH, soluble ubiquinol, tetrahydrobiopterin, alpha-ketoglutaric acid, carnitine, and/or alpha-lipoic acid, nutritional co-factors, calcium beta-methyl-beta-hydroxybutyrate, arginine alpha-ketoglutarate, sodium R-alpha lipoic acid, thiamine, riboflavin, niacin, pyridoxine, ascorbic acid, citric acid, malic acid, sodium benzoate, potassium sorbate, acesulfame K, aspartame, xanthan gum, or a combination thereof. Non-limiting examples of nutritional co-factors include R-alpha lipoic acid, acetyl-1-carnitine, ketoisocaproate, alpha-ketoglutarate, alpha-hydroxyisocaproate, creatine, branched chain amino acids (such as leucine, isoleucine, and valine), beta-hydroxy-beta methylbutyrate (HMB), B vitamins, vitamin C, soluble ubiquinol, and carnitine.

In some embodiments, the ketogenic composition can be in a variety of forms. For example, the ketogenic composition can be in solid form, semi-solid form, or liquid dosage forms. The ketogenic composition can be in the form of tablets, pills, powders, liquid solutions or suspensions, suppositories, and injectable or infusible solutions. The preferred form depends on the intended mode of administration and therapeutic application.

In some embodiments, the ketogenic composition can be a solid, for example a powder, tablet, gel, bar, confectionary product, or granule, and intended for use as a solid oral dose form. The solid composition can be mixed before use with a liquid, such as a water-based liquid (e.g., fruit drink, dairy product, milk, or yogurt), to provide a liquid drink for the user. The ketogenic composition can be provided, as desired, as a liquid product in a form ready for consumption or as a concentrate or paste suitable for dilution on use. The liquid product can be pH adjusted with citric and/or malic acid, and artificial sweetener and flavoring can be added. The liquid product can be homogenized and pasteurized. The ketogenic composition can further include a pharmaceutically acceptable excipient, diluent, or carrier.

The ketogenic composition can further include a pharmaceutically acceptable carrier or excipient. Such carriers may be sterile liquids, such as water and oils. For example, the carrier an be petroleum oil such as mineral oil; vegetable oil such as peanut oil, soybean oil, or sesame oil; animal oil; or oil of synthetic origin. Suitable carriers can also include ethanol, dimethyl sulfoxide, glycerol, silica, alumina, starch, sorbitol, inositol, xylitol, D-xylose, mannitol, powdered cellulose, microcrystalline cellulose, talk, colloidal silicon dioxide, calcium carbonate, magnesium carbonate, calcium phosphate, calcium aluminum silicate, aluminum hydroxide, sodium starch phosphate, lecithin, and equivalent carriers and diluents. Saline solutions and aqueous dextrose and glycerol solutions can also be employed as liquid carriers. Suitable carriers can include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The ketogenic composition can contain minor amounts of wetting and emulsifying agents. The ketogenic composition can contain pH buffering agents.

In some embodiments, the ketogenic composition can be delivered to the subject in any dose sufficient to achieve the desired therapeutic effect. For example, the ketogenic composition can be administered in a dosage range of 1 mg ketogenic compound/kg of body weight to 100 g ketogenic compound/kg body weight. A therapeutically effective amount of a ketogenic compound can be 1 mg ketogenic compound/kg body weight to 25,000 mg/kg, 5 mg/kg to 10,000 mg/kg, 10 mg/kg to 5,000 mg/kg, 15 mg/kg to 1,000 mg/kg, 20 mg/kg to 800 mg/kg, 25 mg/kg to 750 mg/kg, 30 mg/kg to 700 mg/kg, 35 mg/kg to 650 mg/kg, 40 mg/kg to 600 mg/kg, 45 mg/kg to 550 mg/kg, 50 mg/kg to 500 mg/kg, 55 mg/kg to 450 mg/kg, 60 mg/kg to 400 mg/kg, 65 mg/kg to 350 mg/kg, 70 mg/kg to 300 mg/kg, 75 mg/kg to 250 mg/kg, 80 mg/kg to 200 mg/kg, 85 mg/kg to 150 mg/kg, and 90 mg/kg to 100 mg/kg. A therapeutically effective amount of a ketogenic compound can be 1.25 mg/kg, 2.5 mg/kg, 5 mg/kg, 10 mg/kg, 50 mg/kg, 100 mg/kg, 500 mg/kg, 1,000 mg/kg, 1,500 mg/kg, 2,000 mg/kg, 2,500 mg/kg, 3,000 mg/kg, 3,500 mg/kg, 4,000 mg/kg, 4,500 mg/kg, 5,000 mg/kg, 7,500 mg/kg, 10,000 mg/kg, 25,000 mg/kg, 50,000 mg/kg, or 100,000 mg/kg.

The ketogenic composition can be administered in various ways, including, for example, orally, intragastrically, or parenterally (referring to intravenously and intra-arterially and other appropriate parenteral routes), among others. Administration of the ketogenic composition can be as a single dose, or multiple doses over a period of time. The ketogenic composition can be administered to the patient at any frequency necessary to achieve the desired therapeutic effect. For example, the ketogenic composition can be administered once to several times every month, every two weeks, every week, or every day. Administration of the ketogenic composition can be repeated until the desired therapeutic effect has been achieved. For example, the ketogenic composition can be administered once to several times over the course of 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, or more.

The amount of composition administered will depend on absorption, distribution, metabolism, and excretion rates of the components of the composition as well as other factors known to those of skill in the art. Dosage values may also vary with the severity of the symptoms to be alleviated. The compositions may be administered once, or may be divided and administered over intervals of time. It is to be understood that administration may be adjusted according to individual need and profession judgement of a person administering or supervising the administration of the compositions used herein.

The dose of the composition administered to the subject may vary with the particular components of the composition, the method of administration, and the particular symptoms desired to be alleviated. The dose should be sufficient to affect a desirable response, such as the mitigation, attenuation, or amelioration of at least one symptom of Kabuki syndrome.

The dose of composition administered may be based on the levels of circulating glucose and/or ketone bodies measured in the subject. The levels of circulating glucose and ketone bodies may be measured in a subject prior to or following administration a ketogenic composition. Circulating levels may be determined from, for example, bodily fluids (e.g. blood, serum, plasma, or urine) or breath (such as, acetone on the breath). Any suitable measuring device or kit known in the art may be used, such as the PRECISION XTRA® blood glucose and ketone monitoring kit (Abbott Laboratories, Abbott Park, IL).

In some embodiments, the method comprises administering to the subject the ketogenic composition described herein in combination or alternation with an additional therapeutic agent.

Kits for practicing the methods described herein are further provided. By “kit” is intended any manufacture (e.g., a package or a container) comprising at least one reagent, e.g., any one of the ketogenic compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a ketogenic composition comprising a ketogenic compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more ketogenic compounds, such as those agents described herein. In one embodiment, a kit includes instructions or packaging materials that describe how to administer a ketogenic compound or composition of the kit. Containers of the kit can be of any suitable material, e.g., glass, plastic, metal, etc., and of any suitable size, shape, or configuration. In one embodiment, a ketogenic composition disclosed herein is provided in the kit as a solid, such as a tablet, pill, or powder form. In another embodiment, a ketogenic composition disclosed herein is provided in the kit as a liquid or solution. In one embodiment, the kit comprises an ampoule or syringe containing a ketogenic composition disclosed herein in liquid or solution form.

The following further embodiments of the present disclosure are as provided below:
Embodiment 1. A method of treating one or more symptoms of Kabuki Syndrome in a subject in need thereof, the method comprising administering a therapeutically effective amount of a ketogenic composition comprising a ketogenic compound.
Embodiment 2. A method to attenuate, mitigate, or ameliorate one or more symptoms of Kabuki Syndrome in a subject in need thereof, the method comprising administering a therapeutically effective amount of a ketogenic composition comprising a ketogenic compound.
Embodiment 3. The method of embodiment 1 or embodiment 2, wherein the subject is a human.
Embodiment 4. The method of any one of embodiments 1-3, wherein the Kabuki Syndrome is Type 1 Kabuki Syndrome.
Embodiment 5. The method of any one of embodiments 1-3, wherein the Kabuki Syndrome is Type 2 Kabuki Syndrome.
Embodiment 6. The method of any one of embodiments 1-5, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.
Embodiment 7. The method of any one of embodiments 1-6, wherein the ketogenic compound comprises a ketone ester, a ketone salt, a ketone body precursor, or a combination thereof.
Embodiment 8. The method of any one of embodiments 1-7, wherein the ketogenic compound comprises a beta-hydroxybutyrate (BHB).
Embodiment 9. The method of any one of embodiments 1-8, wherein the ketogenic composition further comprises a medium chain triglyceride (MCT).
Embodiment 10. The method of embodiment 9, wherein the ketogenic compound and the MCT are present at about a 1:1 ratio.
Embodiment 11. The method of embodiment 9 or embodiment 10, wherein the ketogenic compound and the MCT are present in an amount from about 5% to about 25% by weight of the composition.
Embodiment 12. The method of any one of embodiments 9 to 11, wherein the ketogenic compound and the MCT are present in an amount of about 15% by weight of the composition.
Embodiment 13. A method of treating one or more symptoms of Kabuki Syndrome in a subject in need thereof comprising administering a therapeutically effective amount of a ketogenic composition to the subject, wherein the ketogenic composition comprises a beta-hydroxybutyrate (BHB) and a medium chain triglyceride (MCT).
Embodiment 14. The method of embodiment 13, wherein the BHB and the MCT are present at about a 1:1 ratio.
Embodiment 15. The method of embodiment 13 or embodiment 14, wherein treating one or more symptoms of Kabuki Syndrome comprises attenuating, mitigating, or ameliorating the one or more symptoms of Kabuki Syndrome.
Embodiment 16. The method of any one of embodiments 13-15, wherein the subject is a human.
Embodiment 17. The method of any one of embodiments 13-16, wherein the Kabuki Syndrome is Type 1 Kabuki Syndrome.
Embodiment 18. The method of any one of embodiments 13-16, wherein the Kabuki Syndrome is Type 2 Kabuki Syndrome.
Embodiment 19. The method of any one of embodiments 13-18, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.
Embodiment 20. The method of any one of embodiments 13-19, wherein the BHB and the MCT are present in an amount from about 5% to about 25% by weight of the composition
Embodiment 21. The method of any one of embodiments 13-20, wherein the BHB and MCT are present in an amount of about 15% by weight of the composition.
Embodiment 22. A ketogenic composition comprising a ketogenic compound and a medium chain triglyceride (MCT) for use in treating one or more symptoms of Kabuki Syndrome in a subject in need thereof.
Embodiment 23. The ketogenic composition of embodiment 22, wherein the ketogenic compound comprises a beta-hydroxybutyrate (BHB).
Embodiment 24. The ketogenic composition of embodiment 22 or embodiment 23, wherein the ketogenic compound and the MCT are present at about a 1:1 ratio.
Embodiment 25. The ketogenic composition of any one of embodiments 22-24, wherein the Kabuki Syndrome is Type 1 Kabuki Syndrome.
Embodiment 26. The ketogenic composition of any one of embodiments 22-24, wherein the Kabuki Syndrome is Type 2 Kabuki Syndrome.
Embodiment 27. The ketogenic composition of any one of embodiments 22-26, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.
Embodiment 28. The ketogenic composition of any one of embodiments 22-27, wherein the ketogenic compound and the MCT are present in an amount from about 5% to about 25% by weight of the composition
Embodiment 29. The ketogenic composition of any one of embodiments 22-28, wherein the ketogenic compound and MCT are present in an amount of about 15% by weight of the composition.
Embodiment 30. Use of a ketogenic compound and a medium chain triglyceride (MCT) in the manufacture of a ketogenic composition for treating one or more symptoms of Kabuki Syndrome in a subject in need thereof.
Embodiment 31. The use of embodiment 30, wherein the ketogenic compound comprises a beta-hydroxybutyrate (BHB).
Embodiment 32. The use of embodiment 30 or embodiment 31, wherein the ketogenic compound and the MCT are present at about a 1:1 ratio.
Embodiment 33. The use of any one of embodiments 30-32, wherein the Kabuki Syndrome is Type 1 Kabuki Syndrome.
Embodiment 34. The use of any one of embodiments 30-32, wherein the Kabuki Syndrome is Type 2 Kabuki Syndrome.
Embodiment 35. The use of any one of embodiments 30-34, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.
Embodiment 36. The use of any one of embodiments 30-35, wherein the ketogenic compound and the MCT are present in an amount from about 5% to about 25% by weight of the composition
Embodiment 37. The use of any one of embodiments 30-36, wherein the ketogenic compound and MCT are present in an amount of about 15% by weight of the composition.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are put forth so as to provide those of ordinary skill in the art with a complete disclosure and description of how compositions and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

Kabuki Syndrome is a rare genetic disorder that leads to many developmental abnormalities, and is caused by a heterozygous mutation in either KMT2D (Type 1) or KDM6A (Type 2) that leads to a loss of function. Both genes play a role in gene regulation via histone modification. KMT2D is a gene that codes for the protein kmt2d, a lysine methyltransferase responsible for methylating H3K4. The KDM6A gene encodes the protein kdm6a, a demethylase responsible for demethylating H3K27. Both gene functions contribute to the opening of the chromatin. When either of these two proteins are deficient, craniofacial, skeletal, mental, and dermatologic development are severely impacted. Other developmental processes are impacted, but to varying the across patients.

The present example uses a 15% by weight 1:1 BHB and MCT supplementation (i.e. total mixture has 7.5% BHB and 7.5% MCT by weight) in standard rodent chow to epigeneticially modify c57bl/6 and Kmt2d+/Geo mice.

Kmt2d^{+/βGeo} (Type 1 Kabuki syndrome model) mice were fed standard diet with or without 15% BHB-MCT supplementation for 12-15 weeks. Blood ketones, glucose, and body weight were monitored during the treatment period. Open field, Elevated Plus Maze, Novel Object Recognition, and Grip Strength behavioral tests were performed at the end of treatment. Various tissues were harvested, including the brain, for ongoing ex vivo analysis of markers associated with learning, memory, and development.

The untreated Kmt2d^{+/βGeo} mice data show a trend toward lower glucose levels when compared to C57Bl/6j mice. There is also a trend showing that untreated Kmt2d^{+/βGeo} mice having lower body weight than C57Bl/6j mice. Elevated Plus Mazer (EPM) testing showed a trend toward a slight increase in exploratory behavior in the untreated Kmt2d^{+/βGeo} group when compared to C57bl/6j mice. Treated C57Bl/6j mice also showed an increase in exploratory behavior, as well as an increase in brain weight, when compared to untreated C57Bl/6j mice.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method of treating, attenuating, mitigating, or ameliorating one or more symptoms of Kabuki Syndrome in a subject in need thereof, the method comprising administering a therapeutically effective amount of a ketogenic composition comprising a ketogenic compound.

2. (canceled)

3. The method of claim 1, wherein the subject is a human.

4. The method of claim 1, wherein the Kabuki Syndrome is Type 1 Kabuki Syndrome.

5. The method of claim 1, wherein the Kabuki Syndrome is Type 2 Kabuki Syndrome.

6. The method of claim 1, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.

7. The method of claim 1, wherein the ketogenic compound comprises a ketone ester, a ketone salt, a ketone body precursor, or a combination thereof.

8. The method of claim 1, wherein the ketogenic compound comprises a beta-hydroxybutyrate (BHB).

9. The method of claim 1, wherein the ketogenic composition further comprises a medium chain triglyceride (MCT).

10. The method of claim 9, wherein the ketogenic compound and the MCT are present at about a 1:1 ratio.

11. The method of claim 9, wherein the ketogenic compound and the MCT are present in an amount from about 5% to about 25% by weight of the composition.

12. The method of claim 9, wherein the ketogenic compound and the MCT are present in an amount of about 15% by weight of the composition.

13. A method of treating one or more symptoms of Kabuki Syndrome in a subject in need thereof comprising administering a therapeutically effective amount of a ketogenic composition to the subject,
wherein the ketogenic composition comprises a beta-hydroxybutyrate (BHB) and a medium chain triglyceride (MCT).

14. The method of claim 13, wherein the BHB and the MCT are present at about a 1:1 ratio.

15. The method of claim 13, wherein treating one or more symptoms of Kabuki Syndrome comprises attenuating, mitigating, or ameliorating the one or more symptoms of Kabuki Syndrome.

16. The method of claim 13, wherein the subject is a human.

17. The method of claim 13, wherein the Kabuki Syndrome is Type 1 Kabuki Syndrome.

18. The method of claim 13, wherein the Kabuki Syndrome is Type 2 Kabuki Syndrome.

19. The method of claim 13, wherein the one or more symptoms are selected from long eyelashes, depressed nasal tip, atypical fingerprints, ear deformity, hypotonia, joint hyper-flexibility, ptosis, blue sclera, café au lait spot, genitourinary abnormalities, gastrointestinal abnormalities, hearing loss, immune deficiencies, feeding difficulties, obesity, short stature, poor sleep, hyperinsulinemia, hypoglycemia, epilepsy, cardiac defects, vertebral anomalies, sparse lateral eyelash, finger anomalies, cleft palate, dental issues, precocious puberty, scoliosis, and hip dysplasia.

20. The method of claim 13, wherein the BHB and the MCT are present in an amount from about 5% to about 25% by weight of the composition

21. The method of claim 13, wherein the BHB and MCT are present in an amount of about 15% by weight of the composition.

22-37. (canceled)
